(19) United States
(12) Patent Application Publication
(10) Pub. No.: US 2016/0081979 A1
(43) Pub. Date: Mar. 24, 2016

(54) METHOD FOR SEPARATING THERAPEUTIC AGENT FOR CHRONIC MYELOGENOUS LEUKEMIA FROM BARK OF LIRIODENDRON TULIPIFERA L.

(71) Applicant: CHO DANG PHARM. CO., LTD., Seoul (KR)

(72) Inventors: Ki Woon KIM, Seoul (KR); Hye Dong YOO, Seoul (KR); Yong Hwan KIM, Gyeonggi-do (KR); Il Kyong HWANG, Seoul (KR); Young Seon KIM, Incheon (KR); Mi Jung JU, Gyeonggi-do (KR); Su Jin KANG, Gangwon-do (KR)

(73) Assignee: CHO DANG PHARM. CO., LTD., Seoul (KR)

(21) Appl. No.: 14/786,715

(22) PCT Filed: Apr. 23, 2014
(86) PCT No.: PCT/KR2014/003540

§ 371 Date: Oct. 23, 2015

(30) Foreign Application Priority Data
Apr. 26, 2013 (KR) 10-2013-0046505

(51) Int. Cl. A61K 31/365; A61K 36/57

(52) U.S. Cl. CPC A61K 31/365; A61K 36/57; A61K 2236/333; A61K 2236/39; A61K 2236/15

(57) ABSTRACT

The present invention provides a method for separating and preparing a therapeutic agent for chronic myelogenous leukimeia capable of effectively inhibiting growth of T315I, which is known as a mutation of a BCR-ABL fusion gene that causes chronic myelogenous leukemia, comprising the steps of: 1) preliminary extracting by adding ethyl acetate to finely chopped bark of Liriodendron tulipifera L., adding butanol to an extracted fluid for layer-separating an ethyl acetate layer and a butanol layer, removing the butanol layer, and decompression-condensing the remainder to obtain a crude extract; and 2) adding a C1-C3 lower alcohol aqueous solution and n-hexane to the obtained crude extract, removing lipids and water insoluble material dissolved in the n-hexane layer, and obtaining and separating a lower alcohol aqueous solution layer to separate and refine highly pure epi-tulipinolide and costunolide.

### TECHNICAL FIELD

The present invention relates to a process for preparing the extract containing epi-tulipinolide and costunolide as active ingredient from bark of Liriodendron tulipifera for treating chronic myelogenous leukemia. Further, the present invention also relates to a therapeutic agent for treating chronic myelogenous leukemia containing epi-tulipinolide and costunolide extracted from bark of Liriodendron tulipifera.

### DESCRIPTION OF PRIOR ART

Liriodendron tulipifera L. is a kind of deciduous broad-leaved arboreal belonged to Magnoliaceae family. In the bark of Liriodendron tulipifera, alkaloid, sesquiterpene and/or lignan have been known to be included.

Further, in the extract of Liriodendron tulipifera, sesquiterpene lactone compounds including costunolide, tulipinolide, epi-tulipinolide, epi-tulipdienolide and/or gamma-liriodenolide have been included.

Further, parthenolide among these sesquiterpene lactones has been known as anti inflammatory and/or anti cancer agent mediating nuclear transcription factor NF-κB, p21 and/or cyclin D1. Particularly, parthenolide and/or LC-1 (dimethylamino-parthenolide) has been known as anti cancer agent for acute myeloid leukemia inducing its apoptosis by suppressing the activity of nuclear transcription factor NF-κB.

It is a form of chronic myeloid leukemia characterized by the increased and unregulated growth of predominantly myeloid cells in the bone marrow and the accumulation of these cells in the blood. Further, the kinds of leukemia can be classified into acute myeloid leukemia, chronic myeloid leukemia, acute lymphocytic leukemia and chronic lymphocytic leukemia.

In case of the chronic myeloid leukemia occupying 15˜20% of total adult leukemia, this disease has been associated with the chromosomal translocation called the Philadelphia chromosome, which increases the unregulated growth of abnormal myeloid cells in the bone marrow. Further, Philadelphia chromosome has been called for a BCR-ABL fusion gene which has been made by translocation of the part of 9th chromosome and the part of 22nd chromosome.

Such BCR-ABL fusion gene can induce the expression of fusion protein showing tyrosine kinase activity, which has been known as involving the p210 protein biosynthesis. Since the biosynthesized p210 protein can inhibit the apoptosis of myeloid progenitor cells, myeloid progenitor cells can be abnormally multiplied, which results in the myeloid leukemia.

Imatinib (marketed as Gleevec) has been used as 1st generation therapeutics for treating myeloid leukemia, which inhibits tyrosine kinase of BCR-ABL protein. However, it has the problem that the patients having Imatinib resistance have appeared.

Fortunately, the 2nd generation therapeutics, such as, Dasatinib, Nilotinib and Bosutinib have been developed, which can overcome the resistance of Imatinib. However, such 2nd generation therapeutics cannot show the sufficient efficacy any longer, due to the occurrence of T315I enzyme variant expressed by mutant gene of BCR-ABL. Therefore, the 3rd generation therapeutics overcoming the handicaps of 1st and 2nd generation therapeutics has been required for effective treatment of chronic myeloid leukemia caused by T315I enzyme variant.

On the other hand, the inventors of present invention have measured and confirmed that epi-tulipinolide from the extract of the bark of Liriodendron tulipifera is efficacious for treating chronic myeloid leukemia (CML) showing the resistance of both 1st generation therapeutics (Imatinib) and 2nd generation therapeutics.

Further, the inventors of present invention have isolated the components including epi-tulipinolide and costunolide showing pharmacological activity for treating chronic myeloid leukemia from the extract of bark of Liriodendron tulipifera.

On the other hand, the inventors of present invention has also disclosed the method for extracting the components including epi-tulipinolide and costunolide for treating gastrointestinal diseases from bark of Liriodendron tulipifera under Korean Patent No. 10-1353306.

Finally, the present invention has been completed by developing a process for preparing the extract containing epi-tulipinolide and costunolide as active ingredient for treating chronic myeloid leukemia from the extract of the bark of Liriodendron tulipifera through the repeated experiments using various extraction solvent, wherein the component of epi-tulipinolide from the extract of the bark of 3˜5 years old Liriodendron tulipifera is efficacious for treating chronic myeloid leukemia (CML) caused by the occurrence of T315I enzyme variant expressed by mutant gene of BCR-ABL showing the resistance of both 1st generation therapeutics (Imatinib) and 2nd generation therapeutics.

### Problem to be Solved

The problem to be solved is to develop a process for preparing the extract containing epi-tulipinolide and costunolide as active ingredient for treating chronic myeloid leukemia from the extract of the bark of Liriodendron tulipifera through the repeated experiments using various extraction solvent, wherein the component of epi-tulipinolide from the extract of the bark of 3˜5 years old Liriodendron tulipifera is efficacious for treating chronic myeloid leukemia (CML) caused by the occurrence of T315I enzyme variant expressed by mutant gene of BCR-ABL showing the resistance of both 1st generation therapeutics (Imatinib) and 2nd generation therapeutics.

### Means for Solving the Problem

The object of present application is to provide a process for preparing the extract containing epi-tulipinolide and costunolide as active ingredient for treating chronic myeloid leukemia from the extract of the bark of Liriodendron tulipifera comprising the steps of:

obtaining a preliminary extract after adding and extracting 1 wt part of chopped and dried bark of Liriodendron tulipifera with 2˜20 wt part of ethyl acetate,

obtaining a crude extract by collecting and concentrating the ethyl acetate layer under reduced pressure, after removing the butanol layer through the separation of ethyl acetate layer and butanol layer upon adding 0.5˜10 wt part of butanol to the obtained preliminary extract,

obtaining a lower alcohol aqueous mixture by removing the lipid and water insoluble materials in n-hexane layer, after adding C1˜C3 lower alcohol aqueous solution and n-hexane to the obtained crude extract, and

obtaining the high purity of extract including epi-tulipinolide and costunolide by isolating and purifying the obtained lower alcohol aqueous mixture,

wherein said obtained extract comprises 20˜50 wt % of epi-tulipinolide and 5˜20 wt % of costunolide, and the extraction is made by at least one selected from enfleurage, percolation, ultra sonic, maceration or reflux.

Further, after the step for obtaining a lower alcohol aqueous mixture, said method further comprises the step for adding water to the lower alcohol aqueous mixture to obtain the low concentration of lower alcohol aqueous layer; the step for separating dichloromethane layer by adding dichloromethane to the lower alcohol aqueous layer; and the step for isolating, concentrating and drying the material dissolved in dichloromethane layer.

On the other hand, the another object of present application is to provide a therapeutics for treating chronic myeloid leukemia containing epi-tulipinolide and costunolide as active ingredient from the extract of the bark of Liriodendron tulipifera obtained by according to the method described above.

The further object of present application is to provide pharmaceutical composition for treating chronic myeloid leukemia comprising epi-tulipinolide and costunolide as active ingredients as described, and pharmaceutically acceptable carriers.

### Advantageous Effect

The outstanding advantageous effect of present application is to afford a process for preparing the extract containing epi-tulipinolide and costunolide as active ingredient for treating chronic myeloid leukemia from the extract of the bark of Liriodendron tulipifera, wherein the component of epi-tulipinolide from the extract is efficacious for treating chronic myeloid leukemia (CML) caused by the occurrence of T315I enzyme variant expressed by mutant gene of BCR-ABL showing the resistance of both 1st generation therapeutics (Imatinib) and 2nd generation therapeutics.

### BRIEF DESCRIPTION OF DRAWINGS

FIG. 1 shows liquid chromatography data of isolated extract from the bark of Liriodendron tulipifera according to Preparation Example 1. Epi-tulipinolide and costunolide have been confirmed to be included.

FIG. 2 shows liquid chromatography data of extract from the bark of Liriodendron tulipifera according to Comparative Preparation Example 1. Epi-tulipinolide and costunolide have been confirmed to be included.

FIG. 3 shows the 1H NMR spectrum to find out the chemical structure of epi-tulipinolide which has been isolated through liquid chromatography from extract of the bark of Liriodendron tulipifera according to Preparation Example 1.

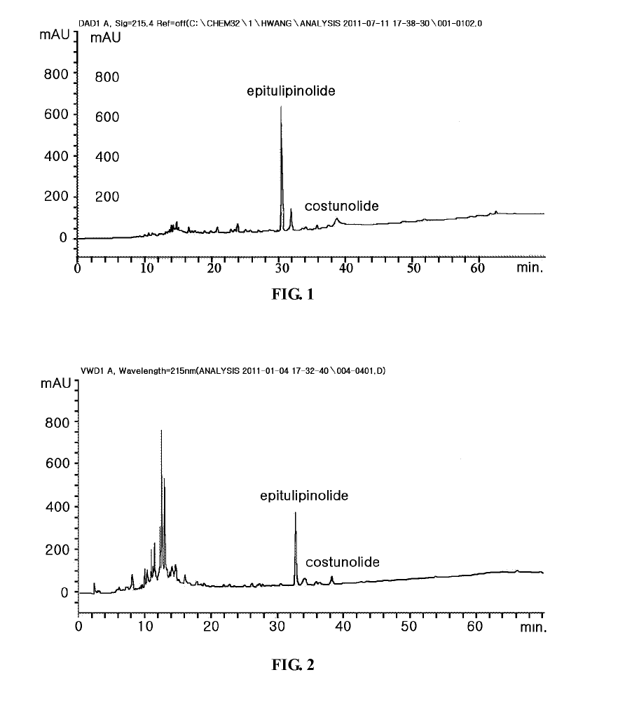

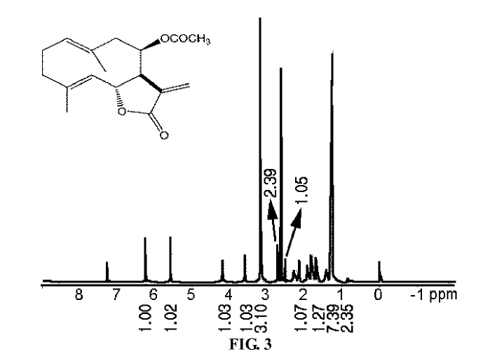

### PREFERRED EMBODIMENT OF INVENTION

The present application affords a process for preparing the extract containing epi-tulipinolide and costunolide as active ingredient for treating chronic myeloid leukemia, wherein the component of epi-tulipinolide from the extract is efficacious for treating chronic myeloid leukemia caused by the occurrence of T315I enzyme variant expressed by mutant gene of BCR-ABL.

Further, the present application affords a process for extracting and isolating the components including epi-tulipinolide and costunolide using ethyl acetate solvent from the bark of 3˜5 years old Liriodendron tulipifera. Therefore, the present application also affords the therapeutic agent for treating chronic myeloid leukemia including epi-tulipinolide and costunolide.

Further, the present application also affords a therapeutic agent containing epi-tulipinolide and costunolide as active ingredient in the extract of the bark of Liriodendron tulipifera for treating chronic myeloid leukemia caused by the occurrence of T315I enzyme variant expressed by mutant gene of BCR-ABL showing the resistance of both 1st generation therapeutics (Imatinib) and 2nd generation therapeutics.

The present application is to provide a process for preparing therapeutic agent containing epi-tulipinolide and costunolide as active ingredient isolated from the extract using ethyl acetate as extraction solvent for treating chronic myeloid leukemia.

Therefore, a therapeutic agent for treating chronic myeloid leukemia contains epi-tulipinolide and costunolide as active ingredient. Of course, epi-tulipinolide and costunolide can be used as standard indicator of the extract from the bark of Liriodendron tulipifera.

Further, the present application is to provide a pharmaceutical composition containing epi-tulipinolide and costunolide as active ingredient isolated from the extract for treating chronic myeloid leukemia.

The present application is to provide a process for preparing a therapeutic agent for treating chronic myeloid leukemia comprising the steps of: (1) obtaining a crude extract from the ethyl acetate layer, after removing the butanol layer upon adding butanol to the preliminary extract, which has been extracted using ethyl acetate from the bark of Liriodendron tulipifera (step 1); and (2) obtaining the isolated extract including epi-tulipinolide and costunolide by isolating and purifying the obtained crude extract (step 2).

In step 1, a crude extract from the ethyl acetate layer has been obtained after removing the butanol layer through the separation of ethyl acetate layer and butanol layer upon adding butanol to the preliminary extract, wherein the preliminary extract is obtained after adding and extracting chopped and dried bark of Liriodendron tulipifera using ethyl acetate as extraction solvent.

In step 2, the high purity of extract including epi-tulipinolide and costunolide has been obtained by isolating and purifying the obtained lower alcohol aqueous mixture, wherein the lower alcohol aqueous mixture is obtained by removing the lipid and water insoluble materials in n-hexane layer, after adding C1˜C3 lower alcohol aqueous solution and n-hexane to the crude extract obtained in step 1.

After the step for obtaining a lower alcohol aqueous mixture, the preparation method of present application can require further 3 steps comprising the step for adding water to the lower alcohol aqueous mixture to obtain the low concentration of lower alcohol aqueous layer; the step for separating dichloromethane layer by adding dichloromethane to the lower alcohol aqueous layer; and the step for isolating, concentrating and drying the material dissolved in dichloromethane layer.

The bark of Liriodendron tulipifera has been collected from the Liriodendron tulipifera located Gangjin-gun, Jeollanam-do in Republic of Korea. After chopping and powdering the collected bark of Liriodendron tulipifera, extraction solvent has been added in a 10˜20 times amount of collected bark. The extraction has been carried out for 24˜96 hours at room temperature to 50 degree. Finally, the extract has been obtained after concentrating and drying it at reduced pressure.

The present invention provides a method for extracting and purifying epi-tulipinolide and costunolide as represented by following formulas from the bark of Liriodendron tulipifera.

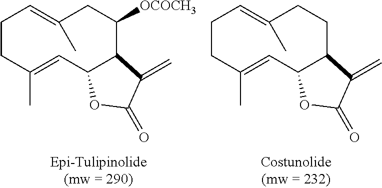

The extract obtained from the bark of Liriodendron tulipifera has been analyzed for measuring epi-tulipinolide (retention time-31 minutes) and costunolide (retention time-36 minutes) amount using Alliance e2695 system according to high performance liquid chromatography method. The conditions for HPLC analysis are as follows. Reverse phase column Kromasil C18, 5 mg/ml of sample concentration, 215 nm of UV wave length (flow rate: 1.0 ml/min), mobile phase gradient condition has been shown in Table 1.

TABLE 1
Mobile phase gradient condition for HPLC analysis

| retention time (min.) | water (wt %) | methanol (wt %) |
|---|---|---|
| 0 | 98 | 2 |
| 10 | 50 | 50 |
| 60 | 0 | 100 |
| 70 | 0 | 100 |
| 80 | 98 | 2 |

As a result of HPLC analysis according to gradient condition as shown in Table 1, the main components of epi-tulipinolide and costunolide have been detected at 31 minute and at 36 minute of retention time respectively. Further, to confirm the presence of epi-tulipinolide and costunolide respectively, each retention time of the standard of epi-tulipinolide and the standard of costunolide has been measured as the same manner. Upon comparing the retention time between standard and obtained material, we have confirmed that epi-tulipinolide and costunolide have been isolated through HPLC.

In the aspect of stereochemistry, the specific rotation of epi-tulipinolide has been measured. The measured value of epi-tulipinolide ([α]D=+74) from the extract of Liriodendron tulipifera has almost corresponded with reported value of epi-tulipinolide ([α]D=+76). Therefore, the presence of epi-tulipinolide has been confirmed by stereochemistry.

On the other hand, each molecular weight of epi-tulipinolide, costunolide and other components from the extract of Liriodendron tulipifera has been measured according to LC-MS. It has been confirmed that the molecular weight of epi-tulipinolide is 290, while the molecular weight of costunolide is 232. Further, other components, ridentin (Mw=264) and deacetyllipiferolide (Mw=264) have been also measured and confirmed.

Further, the chemical structure of epi-tulipinolide has been also confirmed through NMR analysis. Further, the presence of costunolide has been also confirmed using HPLC analysis by comparing the standard costunolide with obtained costunolide extract.

LC-MS analysis instrument made by Waters has been employed using reverse phase column. The measurement has been carried out using 2 μl of sample volume concentration, through 200˜500 nm of UV wave length (flow rate: 0.3 ml/min) The mobile phase gradient condition has been shown in Table 2.

TABLE 2
Mobile phase gradient condition for LC-MS analysis

| retention time (min.) | water (wt %) | ACN (wt %) |
|---|---|---|
| 0 | 85 | 15 |
| 10 | 30 | 70 |
| 60 | 0 | 100 |
| 70 | 0 | 100 |

Further, the obtained extract of the bark of Liriodendron tulipifera of present application comprises 20˜50 wt % of epi-tulipinolide and 5˜20 wt % of costunolide, and the extraction is made by at least one selected from enfleurage, percolation, ultra sonic, maceration or reflux.

### Preparation Example 1

### Preparation of Extract from Bark of Liriodendron tulipifera Using Ethyl Acetate Solvent

1. Solvent Extraction Step

200 g of dried and chopped bark of 3˜5 years old Liriodendron tulipifera has been agitated and extracted using 2000 ml of ethyl acetate at room temperature for 24 hours. After concentrating and filtering the extracted mixture at reduced pressure, the preliminary extract has been obtained. The obtained preliminary extract has been further mixed and extracted using 500 ml of butanol at room temperature for 2˜4 hours. Then, the butanol soluble materials have been removed by separating and removing the butanol layer. Finally, 12.54 g of crude extract has been obtained after concentrating the remained mixture.

2. Purification Step

After dissolving 12.54 g of obtained crude extract with 200 ml of 70% ethanol, 200 ml of n-hexane has been added and agitated to the crude extract mixture. After separating and removing the n-hexane layer, 70% ethanol layer has been obtained. Finally, 2.75 g of purified extract has been obtained upon concentration and freeze drying the obtained mixture.

The amount of epi-tulipinolide and costunolide has been measured using HPLC and LC-MS. Finally, 44.1 wt % of epi-tulipinolide and 15.6 wt % of costunolide have been measured and confirmed.

### Preparation Example 2

### Preparation of Extract from Bark of Liriodendron tulipifera Using Ethyl Acetate Solvent

1. Solvent Extraction Step

200 g of dried and chopped bark of 3˜5 years old Liriodendron tulipifera has been agitated and extracted using 2000 ml of ethyl acetate at room temperature for 72 hours. After concentrating and filtering the extracted mixture at reduced pressure, the preliminary extract has been obtained. The obtained preliminary extract has been further mixed and extracted using 500 ml of butanol at room temperature for 2˜4 hours. Then, the butanol soluble materials have been removed by separating and removing the butanol layer. Finally, 13.72 g of crude extract has been obtained after concentrating the remained mixture.

2. Purification Step

After dissolving 13.72 g of obtained crude extract with 200 ml of 70% ethanol, 200 ml of n-hexane has been added and agitated to the crude extract mixture. After separating and removing the n-hexane layer, 70% ethanol layer has been obtained. Finally, 2.98 g of purified extract has been obtained upon concentration and freeze drying the obtained mixture.

The amount of epi-tulipinolide and costunolide has been measured using HPLC and LC-MS. Finally, 46.2 wt % of epi-tulipinolide and 12.0 wt % of costunolide have been measured and confirmed.

### Preparation Example 3

### Preparation of Extract from Bark of Liriodendron tulipifera Using Ethyl Acetate Solvent

1. Solvent Extraction Step

200 g of crushed bark of 3˜5 years old Liriodendron tulipifera has been agitated and extracted using 2000 ml of ethyl acetate at room temperature for 96 hours. After concentrating and filtering the extracted mixture at reduced pressure, the preliminary extract has been obtained. The obtained preliminary extract has been further mixed and extracted using 500 ml of butanol at room temperature for 2˜4 hours. Then, the butanol soluble materials have been removed by separating and removing the butanol layer. Finally, 25.71 g of crude extract has been obtained after concentrating the remained mixture.

2. Purification Step

After dissolving 25.71 g of obtained crude extract with 200 ml of 70% ethanol, 200 ml of n-hexane has been added and agitated to the crude extract mixture. After separating and removing the n-hexane layer, 70% ethanol layer has been obtained. Finally, 3.84 g of purified extract has been obtained upon concentration and freeze drying the obtained mixture.

The amount of epi-tulipinolide and costunolide has been measured using HPLC and LC-MS. Finally, 47.0 wt % of epi-tulipinolide and 15.4 wt % of costunolide have been measured and confirmed.

### Comparative Preparation Example 1

### Preparation of Extract from Bark of Liriodendron tulipifera Using Chloroform Solvent

1. Solvent Extraction Step

200 g of crushed bark of 3˜5 years old Liriodendron tulipifera has been agitated and extracted using 2000 ml of chloroform at room temperature for 24 hours. After concentrating and filtering the extracted mixture at reduced pressure, the preliminary extract has been obtained. The obtained preliminary extract has been further mixed and extracted using 500 ml of butanol at room temperature for 2˜4 hours. Then, the butanol soluble materials have been removed by separating and removing the butanol layer. Finally, 10.43 g of crude extract has been obtained after concentrating the remained mixture.

2. Purification Step

After dissolving 10.43 g of obtained crude extract with 200 ml of 70% ethanol, 200 ml of n-hexane has been added and agitated to the crude extract mixture. After separating and removing the n-hexane layer, 70% ethanol layer has been obtained. Finally, 1.92 g of purified extract has been obtained upon concentration and freeze drying the obtained mixture.

The amount of epi-tulipinolide and costunolide has been measured using HPLC and LC-MS. Finally, 22.1 wt % of epi-tulipinolide and 5.2 wt % of costunolide have been measured and confirmed.

### Comparative Preparation Example 2

### Preparation of Extract from Bark of Liriodendron tulipifera Using Crude Ethanol Solvent

1. Solvent Extraction Step

200 g of dried and chopped bark of 3˜5 years old Liriodendron tulipifera has been agitated and extracted using 2000 ml of crude ethanol at room temperature for 24 hours. After concentrating and filtering the extracted mixture at reduced pressure, the preliminary extract has been obtained. The obtained preliminary extract has been further mixed and extracted using 500 ml of butanol at room temperature for 2˜4 hours. Then, the butanol soluble materials have been removed by separating and removing the butanol layer. Finally, 19.5 g of crude extract has been obtained after concentrating the remained mixture.

2. Purification Step

After dissolving 19.5 g of obtained crude extract with 200 ml of 70% ethanol, 200 ml of n-hexane has been added and agitated to the crude extract mixture. After separating and removing the n-hexane layer, 70% ethanol layer has been obtained. Finally, 2.65 g of purified extract has been obtained upon concentration and freeze drying the obtained mixture.

The amount of epi-tulipinolide and costunolide has been measured using HPLC and LC-MS. Finally, 15.4 wt % of epi-tulipinolide and 3.7 wt % of costunolide have been measured and confirmed.

### Comparative Preparation Example 3

### Preparation of Extract from Bark of Liriodendron tulipifera Using Crude Ethanol/Water (1:1) Mixed Solvent

1. Solvent Extraction Step

200 g of crushed bark of 3˜5 years old Liriodendron tulipifera has been agitated and extracted using 2000 ml of crude ethanol/water (1:1) mixed solvent at room temperature for 24 hours. After concentrating and filtering the extracted mixture at reduced pressure, the preliminary extract has been obtained. The obtained preliminary extract has been further mixed and extracted using 500 ml of butanol at room temperature for 2˜4 hours. Then, the butanol soluble materials have been removed by separating and removing the butanol layer. Finally, 25.17 g of crude extract has been obtained after concentrating the remained mixture.

2. Purification Step

After dissolving 25.17 g of obtained crude extract with 200 ml of 70% ethanol, 200 ml of n-hexane has been added and agitated to the crude extract mixture. After separating and removing the n-hexane layer, 70% ethanol layer has been obtained. Finally, 1.75 g of purified extract has been obtained upon concentration and freeze drying the obtained mixture.

The amount of epi-tulipinolide and costunolide has been measured using HPLC and LC-MS. Finally, 8.8 wt % of epi-tulipinolide and 2.7 wt % of costunolide have been measured and confirmed.

TABLE 3
The amount of epi-tulipinolide and costunolide in the extract

| | epi-tulipinolide | costunolide |
|---|---|---|
| Prep. Example 1 | 44.1% | 15.6% |
| Prep. Example 2 | 46.2% | 12.0% |
| Prep. Example 3 | 47.0% | 15.4% |
| Com. Prep. Example 1 | 22.1% | 5.2% |
| Com. Prep. Example 2 | 15.4% | 3.7% |
| Com. Prep. Example 3 | 8.8% | 2.7% |

As shown in Table 3, the amount of epi-tulipinolide and costunolide in the extract from the bark of Liriodendron tulipifera according to the method disclosed in Preparation Example 1˜3 has been in the range of 44˜47 wt % and 12˜16 wt % respectively. However, the amount of epi-tulipinolide and costunolide according to Comparative Preparation Example 1˜3 has been in the range of 8˜23 wt % and 2˜6 wt % respectively. Ethyl acetate has been confirmed as the most useful solvent for extracting epi-tulipinolide and costunolide in a high yield.

1. A process for preparing the extract containing epi-tulipinolide and costunolide as active ingredient for treating chronic myeloid leukemia from the extract of the bark of Liriodendron tulipifera comprising the steps of:
obtaining a preliminary extract after adding and extracting 1 wt part of chopped and dried bark of Liriodendron tulipifera with 2˜20 wt part of ethyl acetate,
obtaining a crude extract by collecting and concentrating the ethyl acetate layer under reduced pressure, after removing the butanol layer through the separation of ethyl acetate layer and butanol layer upon adding 0.5˜10 wt part of butanol to the obtained preliminary extract,
obtaining a lower alcohol aqueous mixture by removing the lipid and water insoluble materials in n-hexane layer, after adding C1˜C3 lower alcohol aqueous solution and n-hexane to the obtained crude extract, and
obtaining the high purity of extract including epi-tulipinolide and costunolide by isolating and purifying the obtained lower alcohol aqueous mixture,
wherein said obtained extract comprises 20˜50 wt % of epi-tulipinolide and 5˜20 wt % of costunolide, and the extraction is made by at least one selected from enfleurage, percolation, ultra sonic, maceration or reflux.

2. The process for preparing the extract according to claim 1, after the step for obtaining a lower alcohol aqueous mixture,
said method further comprises the step for adding water to the lower alcohol aqueous mixture to obtain the low concentration of lower alcohol aqueous layer; the step for separating dichloromethane layer by adding dichloromethane to the lower alcohol aqueous layer; and the step for isolating, concentrating and drying the material dissolved in dichloromethane layer.

3. The therapeutics for treating chronic myeloid leukemia containing epi-tulipinolide and costunolide as active ingredient from the extract of the bark of Liriodendron tulipifera obtained by according to the method of claim 1.

4. A pharmaceutical composition for treating chronic myeloid leukemia comprising epi-tulipinolide and costunolide as active ingredients of claim 3, and pharmaceutically acceptable carriers.

5. The therapeutics for treating chronic myeloid leukemia containing epi-tulipinolide and costunolide as active ingredient from the extract of the bark of Liriodendron tulipifera obtained by according to the method of claim 2.

6. A pharmaceutical composition for treating chronic myeloid leukemia comprising epi-tulipinolide and costunolide as active ingredients of claim 5, and pharmaceutically acceptable carriers.
